(19) United States
(12) Patent Application Publication
(10) Pub. No.: US 2013/0006284 A1
(43) Pub. Date: Jan. 3, 2013

(54) OBSTRUCTION REMOVAL ASSEMBLY

(75) Inventors: Steen Aggerholm, St. Heddinge (DK); Bente Weber Christensen, Ringsted (DK); Per Hendriksen, Herlufmagle (DK); Thomas Lysgaard, Solroed Strand (DK); Arne Molgaard-Nielsen, Copenhagen (DK); Frank Svendsen, Ringsted (DK)

(73) Assignee: Cook Medical Technologies LLC, Bloomington, IN (US)

(21) Appl. No.: 13/634,117

(22) PCT Filed: Mar. 10, 2011
(86) PCT No.: PCT/US11/27894

§ 371 Date: Sep. 12, 2012

(30) Foreign Application Priority Data
Mar. 12, 2010 (GB) 10/041267

(51) Int. Cl. A61B 17/22

(52) U.S. Cl. 606/159

(57) ABSTRACT

A clot removal device (10) includes a conical retrieval member (14) affixed to a wire (12) The conical retrieval member (14) has an apex (16) at its distal end, and a base that forms an opening (18) into a cavity The conical retrieval member (14) is formed from a thin sheet of Nitinol film, and expands into its conical configuration at body temperature The clot removal device (10) can be used physically to withdraw a blood clot (40) from a blood vessel (42) without requiring aspiration The member (14) can be pulled by a cord (90) to cause it to rewrap onto itself, in so doing to capture an obstruction such as a thrombus.

### TECHNICAL FIELD

The present invention relates to an obstruction removal assembly. The assembly may be used for the physical removal of thrombi from blood vessels.

### BACKGROUND ART

Mechanical thrombectomy is a procedure that has been in widespread use for many years. Typical thrombectomy devices are balloons that are inflated in a vessel and then withdrawn to pull clots into a sheath which can be withdrawn from the patient to remove the clots. Other devices are simple open ended catheters into which a clot is aspirated and removed from the patient. Another thrombectomy device employs a basket device that is opened within the clot so that the clot becomes captured in the basket. The basket can then be retrieved along with the clot. Still other devices use a small corkscrew shaped device that is collapsed inside a catheter. The catheter is passed through the clot, the corkscrew is pushed out of the catheter allowing the device to expand, capturing the clot for removal. Some corkscrew devices are simply “screwed” into the clot, then retracted into a catheter for removal before the corkscrew is retracted.

U.S. Pat. No. 6,511,492, U.S. Pat. No. 6,669,721, U.S. Pat. No. 6,692,509, U.S. Pat. No. 7,316,692 and U.S. 2005/0283166 disclose various designs of prior art clot removal devices.

Aspirating the clot into a catheter can cause breaking up of the clot during the procedure. Furthermore, where a clot is located in a very narrow vessel, the size of the catheter is too small, and thus it is not practical to remove a clot by this method. Corkscrew devices that are screwed into the clot usually have a smooth rounded tip to prevent the corkscrew from penetrating the vessel wall or otherwise damaging the vessel wall as it is screwed into the clot. With these devices, however, the smooth, rounded central tip does not screw into the clot, but instead is pushed into the clot and then the remainder of the corkscrew is screwed into the clot. This results in a pushing force on the center of the clot and a pulling force on the periphery of the clot. These counter forces tend to macerate or fragment the clot and can result in only a small part of the clot being captured. Some corkscrew devices may substitute a sharp tip that can screw directly into the clot for the rounded tip. However, sharp tips can penetrate the vessel wall just as easily as they can penetrate and capture the clot. Such devices are seldom used since they carry the very high risk of penetrating the vessel wall. When a bead or ball is applied to the tip of the device that is large enough to protect the vessel wall, it will be so large that it will tend to push the clot distally rather than penetrate the clot such that the clot can be captured and removed.

Still another issue includes capturing any of the loose fragments possibly dislodged from the clot during removal of the clot. Another issue arises if a thrombectomy device has a diameter smaller than the vessel diameter when deployed from the catheter. In such a case, some of the clot immediately adjacent the vessel wall may not be removed.

Another issue associated with conventional thrombectomy devices is that they are typically too large and too stiff for use in the small tortuous vessels in the brain. Some of the conventional devices also use a central mandrel wire or some other structure for support, which displaces clots, making it difficult to capture all of the clot material.

### DISCLOSURE OF THE INVENTION

The present invention seeks to provide an improved obstruction removal assembly and method.

According to an aspect of the present invention there is provided an obstruction removal assembly for removing an obstruction from a body vessel, including: an elongate catheter member; an expandable retrieval device including a proximal end and a distal end; the retrieval device being movable between a position within the catheter member and an extended position substantially beyond the catheter member; wherein the retrieval device is expandable when in said expanded position into a tapered shape having a chamber for receiving an obstruction, the chamber being open from an access point; said tapered shape forming a smooth taper between the access point of the chamber and the proximal end of the retrieval device and being such that the proximal end of the retrieval device remains inside the catheter member; and a withdrawal mechanism coupled to the retrieval device operable to withdraw the retrieval device into the catheter member.

The withdrawal mechanism operates once the retrieval device has captured an obstruction, in the preferred embodiment a thrombus, to pull the thrombus into the catheter element. Thus, the obstruction can be captured and then held securely in the assembly for complete removal from the patient.

In the preferred embodiment, the retrieval device is expandable by unwrapping to provide a substantially conical trapping member and is rewrappable for withdrawal into the catheter element. Advantageously, the withdrawal mechanism is operable to cause said rewrapping of the retrieval device.

The catheter member may include an internal wall which provides a wrapping force to assist in rewrapping of the retrieval device.

Advantageously, the device is in the form of a film or sheet. Advantageously, the retrieval device is openable to provide an open cone and an portion which extends in a proximal direction beyond the cone, said portion providing for rewrapping to the device upon withdrawal into the catheter element. Said proximally extending portion preferably extends to either side of the device at the point of opening of the cone. The arrangement is such as to provide for the proximally extending portion to assist in the rewrapping or coiling of the device upon its withdrawal into the catheter element.

The film or sheet may be apertured or pierced. The area of the perforations or apertures may be less than 50% of the area of the film or sheet.

In the preferred embodiment, the device is in the form of a quadrilateral sheet of material when viewed flat, most preferably of kite, rhombus or diamond shape. Such a shape allows the retrieval device, including the conical portion and the proximally extending portion to be formed integrally in terms of shape and in particular allows for the proximally extending portion to extend to the extremities of the film or sheet parts which form the open ends of the cone. In this manner, the proximally extending portion can act to provide a rewrapping force by pulling the ends of the film or sheet at the open end of the cone closed again.

In the preferred embodiment, the withdrawal mechanism includes a pulling element operable on the proximal end of the retrieval device to urge the latter into a wrapped configuration. The pulling element is advantageously a cord or wire able to be pulled in a proximal direction.

The arrangement is such as to enable the retrieval device to capture a thrombus or other obstruction and then to close in on itself by rewrapping (or recoiling), whereupon the thrombus is compressed in the retrieval device. Once rewrapped in this manner, the retrieval device can be withdrawn into the catheter element with the thrombus held therewithin. Thus, the thrombus can be removed completely in a safe manner.

Advantageously, the catheter element is an aspiration catheter. As such, the catheter element can assist in the removal of the obstruction from the patient and in particular with the absorption of the obstruction into the catheter element. Moreover, as the aspiration force of such a catheter is largely dependent upon the dimensions of the catheter, that is of its internal cross-sectional area, this arrangement can be used also with small diameter aspiration caterers as the retrieval device will assist in the retrieval of the thrombus.

Advantageously, the retrieval device is formed from a shape memory material, preferably a shape memory metal, alloy or polymer. In the preferred embodiment, the device is formed from Nitinol.

Advantageously, the catheter has an internal diameter of about 1 French (about 0.3 mm) or less.

### BRIEF DESCRIPTION OF THE DRAWINGS

Embodiments of the present invention are described below, by way of example only, with reference to the accompanying drawings, in which:

FIG. 1 shows a perspective view of an exemplary clot removal device in its expanded configuration;

FIG. 2 shows a perspective view of the clot removal device of FIG. 1 in its compressed or wrapped configuration;

FIG. 3 shows a perspective view of the clot removal device of FIG. 1 in a flattened configuration prior to wrapping;

FIGS. 4 to 8 illustrate schematically use of the clot removal device of FIG. 1 to remove a clot from a blood vessel;

FIG. 9 shows a perspective view of a clot removal device in accordance with the present invention, in its expanded configuration; and

FIGS. 12 to 13 show elevational views of the retrieval device of FIG. 9 in various stages of withdrawal into a catheter.

### DETAILED DESCRIPTION

It is to be understood that the Figures are schematic and do not show the various components to their actual scale. In many instances, the Figures show scaled up components to assist the reader in the understanding of the features taught herein.

In this description, the term distal is used to refer to an end of a component which in use is furthest from the surgeon during the medical procedure, including within a patient. The term proximal is used to refer to an end of a component closest to the surgeon and which, in practice, may be in or adjacent an external manipulation part of the deployment or treatment apparatus.

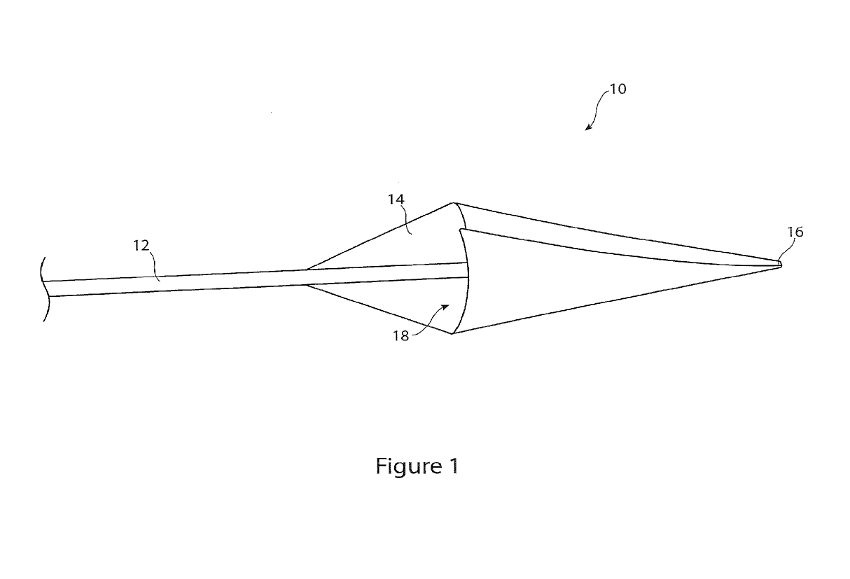

FIG. 1 illustrates an exemplary obstruction removal assembly 10. The removal assembly 10 includes a wire 12 that may be in the form of a guide wire or a similar structure, or which may be of a more substantial rod-like structure, still flexible to be able to pass through a patient's vasculature. At the distal end of the wire 12 is a retrieval device 14 which in this embodiment has a conical form. The retrieval device 14 has an apex 16 at the distal end of the retrieval assembly 10. The base of the forms an opening 18 into the retrieval device 14. The opening 18 is located proximally of the apex 16.

Preferably, the retrieval device 14 is formed from a Nitinol film. Alternatively, any shape memory material, for example an alloy or polymer may be used. The Nitinol film is pre-treated in a known manner so as to be compressible but to revert to a substantially conical expanded configuration at body temperature via a shape memory mechanism, thereby forming the retrieval cone 14 in vivo.

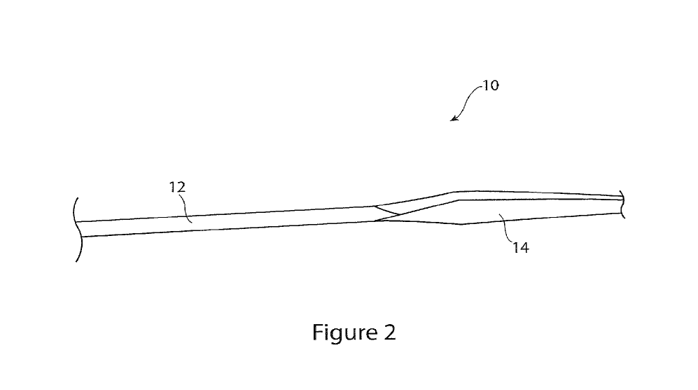

As illustrated in FIG. 1, it can be seen that the walls of the retrieval cone 14 are able to overlap one another and so the cone 14 can be wrapped tightly around itself and around the wire 12 into a compressed configuration (see FIG. 2). When the Nitinol film is below its Austenite finish temperature (for example, at room temperature), the Nitinol is in its martensite form and retains the compressed configuration ready for delivery (see below). Since the Nitinol film is very thin, this arrangement allows a very small compressed configuration to be achieved.

For very small lumen applications, for instance for cerebral applications, the retrieval cone 14 may have a closed diameter able to fit within a micro catheter having an internal diameter of 1 French (0.33 mm) or less. In its expanded configuration (as shown in FIG. 1) the diameter of the retrieval cone 14 at the position of the opening 18 (that is at the base of the cone) may be 1 to 10 mm, for example.

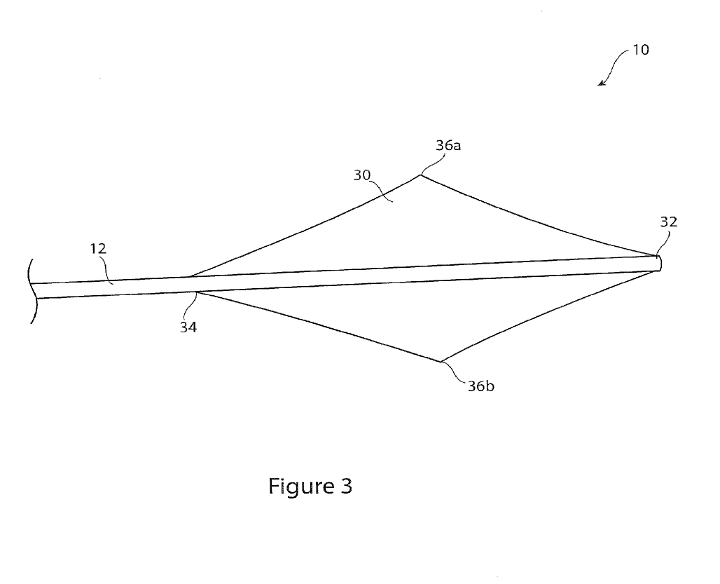

The retrieval cone 14 is preferably formed from a substantially kite, diamond of rhombus shaped sheet of Nitinol film 30, as illustrated in FIG. 3 (hereinafter referred to as kite shaped). The Nitinol film preferably has a length (between the distal end 32, which forms the apex 16 of the retrieval cone and the proximal 34 of the film 30) of 12 mm. The spacing between points 36a and 36b is, in this embodiment, at least 4-6 mm up to around 30 mm or more.

The film preferably has a thickness of 0.1 to 0.5 millimetres.

The Nitinol film is fixed to the wire 12 in any suitable manner, for example, by laser, soldering, crimping, gluing or tying. The skilled person will appreciate that other methods may also be used. The kite-shaped sheet of Nitinol film 30 is formed into a cone as illustrated in FIG. 1 by overlapping the points 36a and 36b. The Nitinol film 30 is treated in a known manner to provide shape memory so that, at body temperature, the Nitinol film 30 is able to form the retrieval cone 14 as described below.

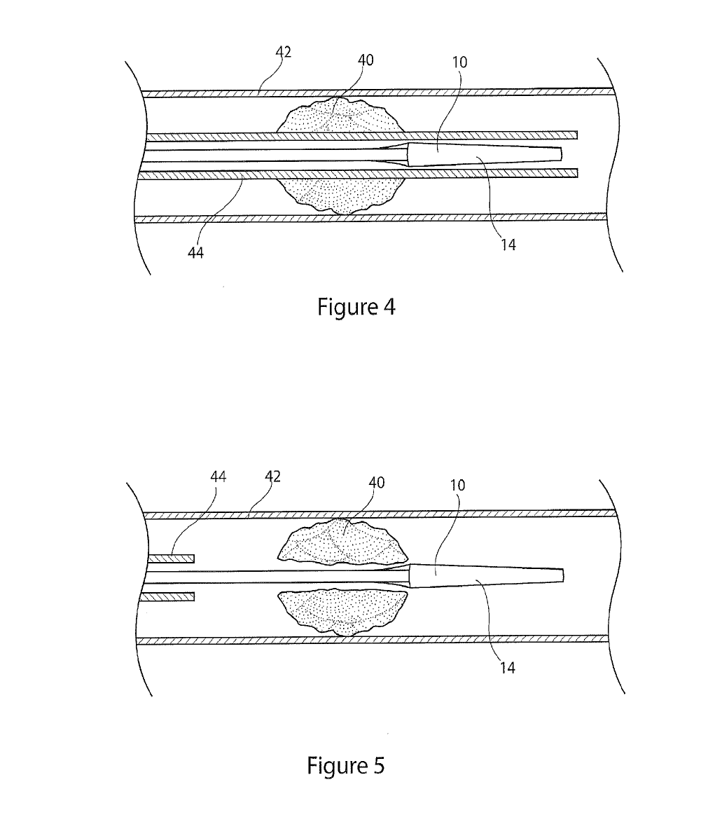

FIGS. 4 to 8 illustrate use of the retrieval device 10 in the removal of a blood clot 40 from a blood vessel 42. As shown in FIG. 4, the device 10 is held within a micro catheter 44 with the retrieval cone 14 in a compressed configuration. The micro catheter preferably has a diameter of less than 1 French (0.33 mm), and so is able to be passed through a thrombus 40 without disturbing the thrombus 40 or otherwise causing the thrombus 40 to break up.

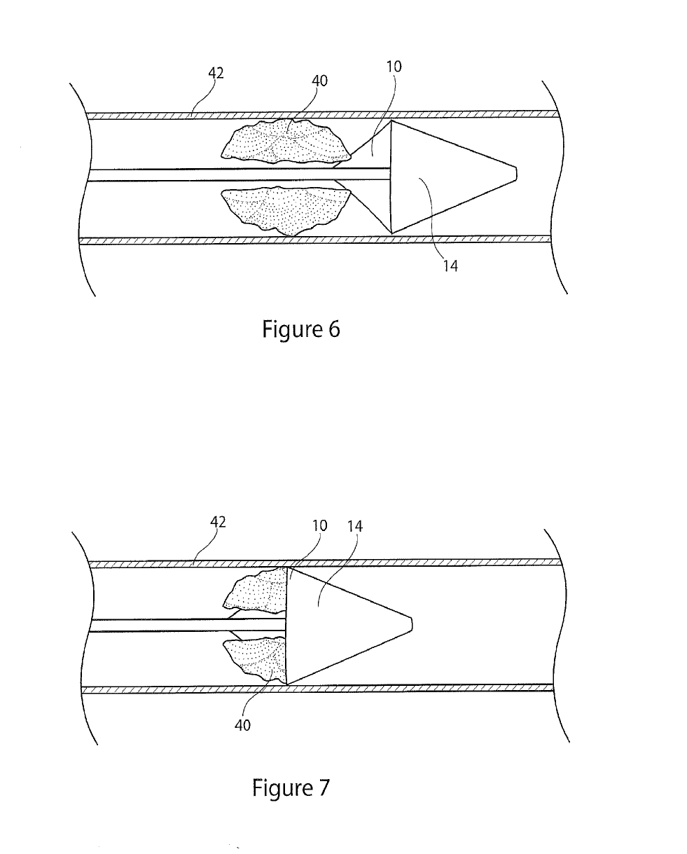

Once the clot removal device 10 has been advanced sufficiently that the proximal end of the retrieval cone 14 is beyond the thrombus 40, the micro catheter 44 is withdrawn (see FIG. 5). The retrieval cone 14, released from the confines of the micro catheter 44, is then able to expand into its shape memory conical form (see FIG. 6). As can be seen, it is preferable that the device 14 is sized such that the open end of the cone extends to the walls of the lumen 42 to be treated.

It will be appreciated that in some instances and for the removal of hard debris such as plaque the device will not necessarily penetrate the thrombus or other obstruction and instead may be fed beyond this to one side of the obstruction or thrombus.

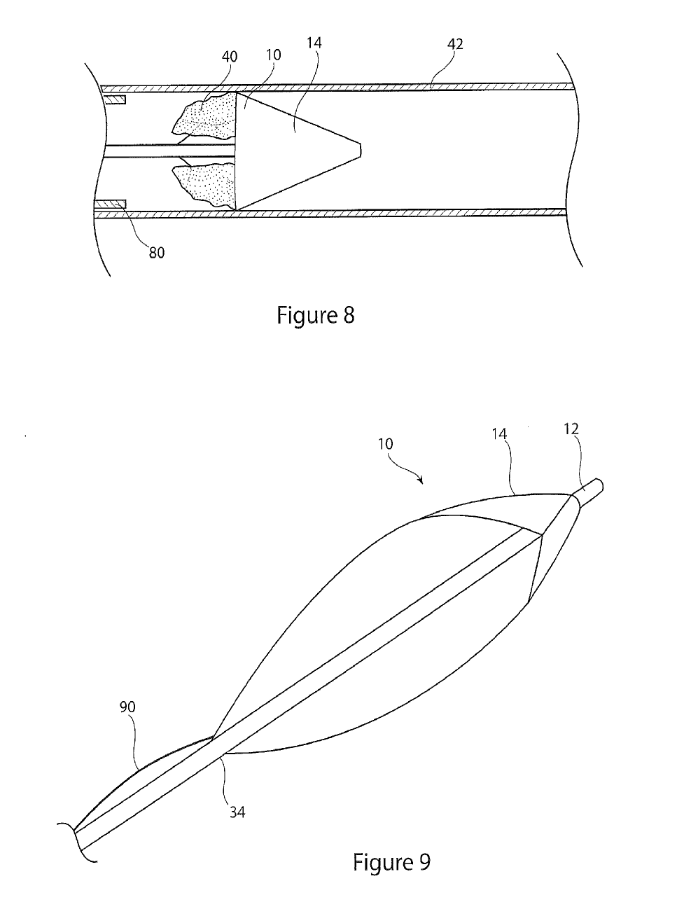

Once the retrieval cone 14 is in its expanded configuration, the device 10 can be pulled back in a proximal direction, as shown in FIG. 7. As this is done, the thrombus 40 is caught within the retrieval cone 14. As the clot retrieval device 10 is further withdrawn proximally, the blood clot 40 is withdrawn therewith (see FIG. 8).

In some examples of the method (and as illustrated in FIG. 8), an aspiration catheter 80 may be introduced into the blood vessel 42 and may be used to assist in removal of the clot 40 from the retrieval cone 14. However, where the blood clot 40 is located in a very narrow vessel, it may be difficult to introduce an aspiration catheter 80, in which case, the blood clot 40 can simply be removed during withdrawal of the clot removal device 10 from the patient. In other words, the clot removal device 10 enables a clot (thrombus) 40 to be removed from a blood vessel 42 without the need for aspiration.

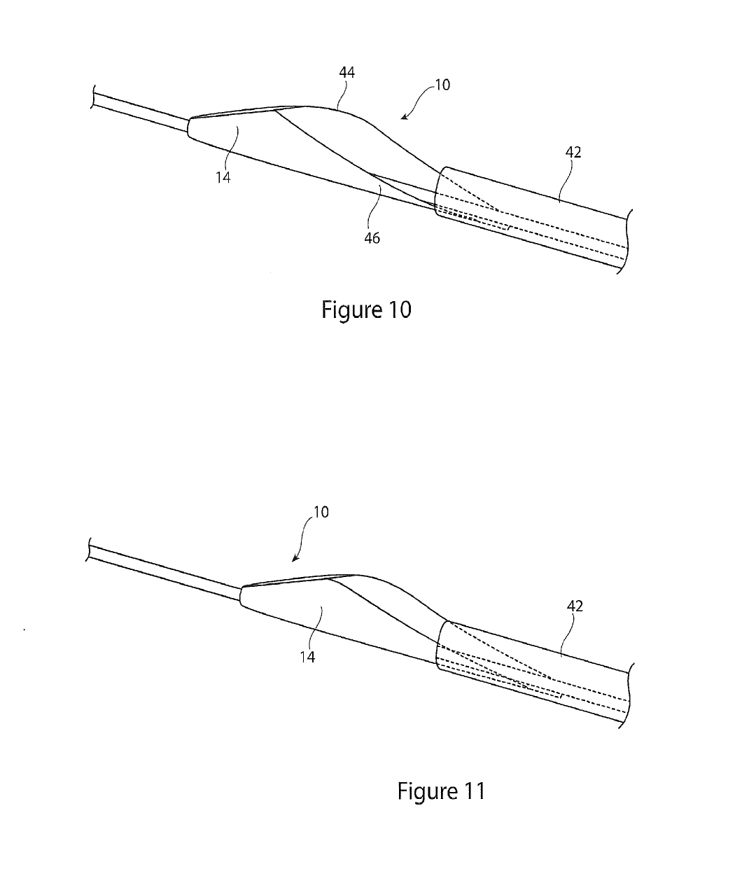

FIG. 9 illustrates an embodiment of obstruction retrieval device 10 in accordance with the present invention. As with the example of FIG. 1, the retrieval device 10 includes a wire or rod 12, which may be of guide wire type, for example. A retrieval cone 14, again preferably formed from a Nitinol film, is attached at its tip or apex to the distal end of the wire 12. The retrieval cone 14 is provided with a withdrawal member 90, which may be a wire, cord or similar structure. The withdrawal member 90 is, in this embodiment, attached to the proximal end 34 of the Nitinol film in any suitable manner, such as by suturing, soldering, crimping or gluing. For this purpose, the proximal end of the device 14 is not fixed to the wire or rod 12 but can slide therealong. In practice, the user can pull the withdrawal member 90 in the proximal direction to cause the retrieval cone 14 to close by rewrapping.

FIGS. 10 to 13 show how the embodiment of FIG. 9 operates in practice.

Referring first to FIG. 10, the device 10 is shown in what could be termed its extended state, in which the device 10 extends beyond the catheter 42 and the device 10 is opened to form the cone 14, which tapers towards the distal end of the assembly 10. In the configuration of FIG. 10, the cone 14 is in a state for catching a thrombus or other obstruction in a body vessel. Typically, the cone 14 at its widest extent is at least as wide as the lumen into which is it intended to be deployed. In practice, the end 44 of the cone 14 may be designed to be wider than the lumen so as to ensure complete spanning of the lumen during the thrombus/obstruction capture operation.

It will be seen in FIG. 10 that the shape of the device 10, that is its kite, diamond or rhombus shape, provides the device 10 with a proximal end 46 which tapers downwardly in the proximal direction of the assembly 10 and at its end into the catheter 42, doing so while presenting a smooth transition in material from the widest portion 44 to the end 34 at the wire or cord 90. This opposing taper assists in the subsequent collapse or rewrap of the device 10 during the thrombus retrieval operation, as described below.

The proximal end 34 of the device 10 remains, in this embodiment, within the catheter 42, retaining a curved radial shape to the proximal end of the device 10, assisting in its subsequent withdrawal into the catheter 42.

Once a thrombus or other obstruction has been caught in the cone 14, the device 10 can be withdrawn into the catheter 42. By pulling on the wire or cord 90, typically in conjunction with pulling into the catheter 42, the device is caused to rewrap on itself. During this process, the thrombus or other obstruction is wrapped into the film forming the device 10 and held therewithin.

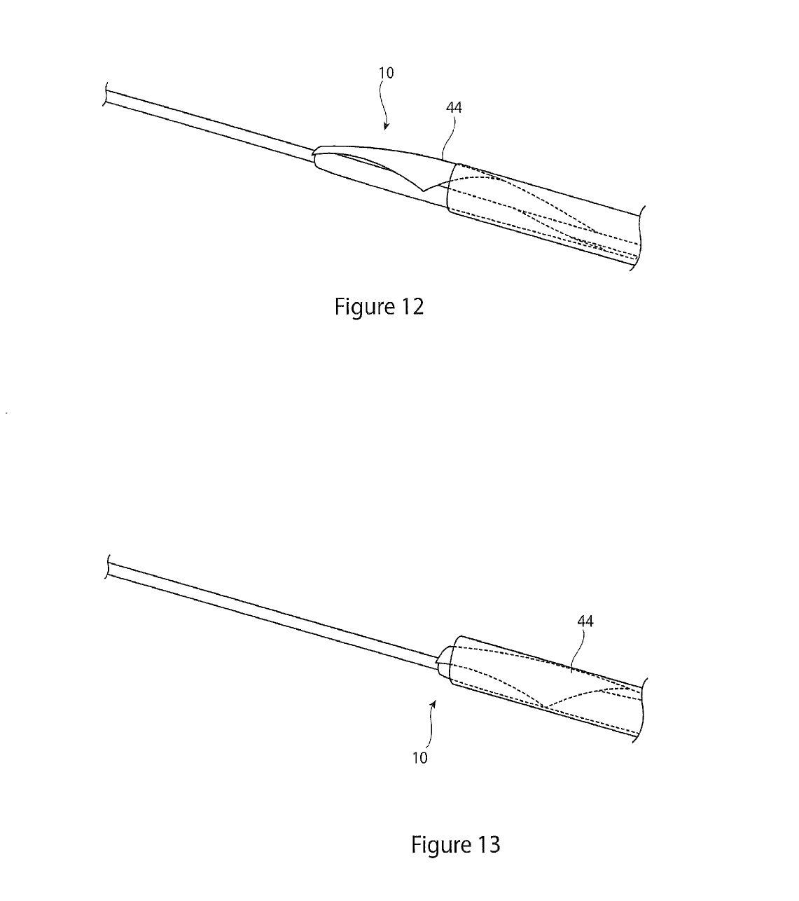

FIGS. 12 and 13 show the device 10 as it is progressively increasingly pulled into the catheter 42 and as it is progressively wrapped tighter. In its final stage, the device 10 is completely wrapped and held within the catheter 42, with the thrombus held therewithin and thus safely captured for removal from within the patient.

In some embodiments, it might be advantageous to use the catheter 42 as an aspiration device, that is to apply aspiration as the device 10 is being pulled to its distal end so as to assist in the retrieval of a thrombus. In other embodiments, there may be provided a second catheter, typically of significantly larger diameter, as an aspiration catheter.

Thus, a surgeon or clinician uses the withdrawal member 90 to pull the retrieval cone 14 in a proximal direction over the wire 12. The configuration of the cone, with its smooth transition or taper between its widest point at the opening of the capture chamber and its proximal end allows the retrieval cone 14 to be compressed, rewrapped or coiled as it is withdrawn into the catheter 42. The retrieval cone 14, the clot 40 and the catheter 42 can then together be withdrawn from the patient.

Although the devices described herein has been described particularly in relation to the removal of blood clots (thrombi), the skilled person will appreciate that the principles disclosed can equally be used in the removal of other types of obstruction from other types of body vessel, such as plaque or other debris.

What have been described and illustrated herein are preferred embodiments of the invention along with some of its variations. The terms, descriptions and Figures used herein are set forth by way of illustration only and are not meant as limitations. Those skilled in the art will recognize that many variations are possible within the scope of the claims, in which all terms are meant in their broadest reasonable sense unless otherwise indicated.

For example, whilst the illustrated embodiments use a non-perforated Nitinol film, the skilled person will appreciate that, in some embodiments, it may be advantageous to form the retrieval cone 14 from a perforated Nitinol film.

The features of the various embodiments described above and their modifications may be substituted for or combined with one another as desired. It is also to be understood that the various features of the dependent claims appended hereto may be used with one another in any desired combination of those claims.

1. An obstruction removal assembly for removing an obstruction from a body vessel, including:
an elongate catheter member;
an expandable retrieval device including a proximal end and a distal end; the retrieval device being movable between a position within the catheter member and an extended position substantially beyond the catheter member; wherein the retrieval device is expandable when in said expanded position into a tapered shape having a chamber for receiving an obstruction, the chamber being open from an access point; said tapered shape forming a smooth taper between the access point of the chamber and the proximal end of the retrieval device and being such that the proximal end of the retrieval device remains inside the catheter member; and
a withdrawal mechanism coupled to the retrieval device operable to withdraw the retrieval device into the catheter member.

2. An assembly according to claim 1, wherein the withdrawal mechanism includes a pulling element for pulling the retrieval device into the catheter element and thereby to pull into the catheter element an obstruction captured in the retrieval device.

3. An assembly according to claim 2, wherein the retrieval device is expandable by unwrapping to provide a substantially conical trapping member and is rewrappable for withdrawal into the catheter element.

4. An assembly according to claim 3, wherein the withdrawal mechanism is operable to cause said rewrapping of the retrieval device.

5. An assembly according to claim 4, wherein said catheter member includes an internal wall which provides a wrapping force to assist in rewrapping of the retrieval device.

6. An assembly according to claim 1, wherein the device is in the form of a film or sheet.

7. An assembly according to claim 6, wherein the film of the retrieval device is openable to provide an open cone and a portion which extends in a proximal direction beyond the cone, said portion providing for rewrapping of the device upon withdrawal into the catheter element.

8. An assembly according to claim 7, wherein the said proximally extending portion extends to either side of the device at the point of opening of the cone.

9. An assembly according to claim 8, wherein the proximally extending portion assists in the rewrapping or coiling of the device upon its withdrawal into the catheter element.

10. An assembly according to [any one of claims 6 to 9] claim 9, wherein the film or sheet is perforated, apertured or pierced.

11. An assembly according to claim 10, wherein the area of the perforations or apertures is less than 50% of the area of the film or sheet.

12. An assembly according to [any one of claims 6 to 11] claim 11, wherein the film is in the form of a quadrilateral sheet of material when viewed flat.

13. An assembly according to claim 12, wherein the film is in the form of one of: a kite, a rhombus and a diamond shape.

14. An assembly according to [any preceding] claim 1, wherein the withdrawal mechanism includes a pulling element able to pull on the proximal end of the retrieval device to urge the latter into a wrapped configuration.

15. An assembly according to claim 14, wherein the pulling element is a cord or wire able to be pulled in a proximal direction.

16. An assembly according to claim 15, wherein the catheter element is an aspiration catheter.

17. An assembly according to claim 1, wherein the retrieval device is formed from a shape memory material.

18. An assembly according to claim 17, wherein the device is formed from one of: a shape memory metal, alloy or polymer.

19. An assembly according to claim 18, wherein the device is formed from Nitinol.

20. An assembly according to claim 1, wherein the catheter has an internal diameter of about 1 French (about 0.3 mm) or less.

21. A method of removing an obstruction from a blood vessel using an obstruction removal assembly provided with: an elongate catheter member; an expandable retrieval device including a proximal end and a distal end; the retrieval device being movable between a position within the catheter member and an extended position substantially beyond the catheter member; wherein the retrieval device is expandable when in said extended position into a tapered shape having a chamber for receiving an obstruction, the chamber being open from an access point; said tapered shape forming a smooth taper between the access point of the chamber and the proximal end of the retrieval device and being such that the proximal end of the retrieval device remains inside the catheter member; and a withdrawal mechanism operable to withdraw the retrieval device into the catheter member;
the method including the steps of: introducing the catheter and the obstruction removal device into a blood vessel of a patient; advancing the catheter and the obstruction removal device through a blood clot to be removed at least until the retrieval device is beyond the obstruction; allowing the retrieval device to expand; moving the retrieval device in a proximal direction to cause the obstruction to enter the chamber of the retrieval device; and withdrawing the retrieval device into the catheter, said withdrawing step causing rewrapping of the retrieval device and catching of the obstruction into the rewrapped device and catheter.

22. A method according to claim 21, including using aspiration via a catheter into the blood vessel of the patient.

23. A method according to claim 22, wherein the obstruction is removed from the patient by fully withdrawing the retrieval device proximally without using aspiration.
